(19) United States
(12) Patent Application Publication
(10) Pub. No.: US 2025/0144162 A1
(43) Pub. Date: May 8, 2025

(54) Composition and Method for Alleviating Bloating and Digestive Symptoms Using Tamarind Seed Powder and Pea Protein Powder

(71) Applicant: Daniel Ricciardi, Austin, TX (US)

(72) Inventor: Daniel Ricciardi, Austin, TX (US)

(21) Appl. No.: 18/882,660

(22) Filed: Sep. 11, 2024

(51) Int. Cl. A61K 36/48; A23L 33/105; A23L 33/185

(52) U.S. Cl. CPC A61K 36/48; A23L 33/105; A23L 33/185

(57) ABSTRACT

The nutritional supplement composition described herein provides a novel and effective means of promoting digestive health and wellness, and from irritable bowel syndrome symptoms such as bloating. Its unique combination of whole tamarind seed powder and pea protein powder taken in an oral formulation offers distinct advantages over existing supplements in the market.

### BACKGROUND

The present invention pertains to a nutritional supplement composition and its method of use. More specifically, the invention involves an oral supplement formulation that combines whole tamarind seed powder and pea protein powder. This composition is designed to offer health benefits to individuals experiencing digestive issues, including bloating.

### SUMMARY OF THE INVENTION

The nutritional supplement composition of the present invention comprises a synergistic blend of whole tamarind seed powder and pea protein powder. This novel formulation, when administered orally, provides superior digestive health benefits compared to conventional supplements.

### DESCRIPTION OF THE INVENTION

The nutritional supplement composition comprises the following ingredients:

Whole Tamarind Seed Powder: Tamarind seed powder is obtained from the seeds of the tamarind tree (Tamarindus indica). The powder contains a high concentration of xyloglucan, a polysaccharide known for its prebiotic and immune-boosting properties. Xyloglucan has been shown to support digestive health, enhance nutrient absorption, and modulate immune function.

Pea Protein Powder: Pea protein powder is derived from yellow peas (Pisum sativum) and is rich in essential amino acids, particularly lysine and arginine. Pea protein is easily digestible and well-tolerated by individuals with dietary sensitivities, such as lactose intolerance or gluten sensitivity.

### METHOD OF PREPARATION

The nutritional supplement composition is prepared by blending tamarind seed powder and pea protein powder to achieve the desired nutritional profile and sensory characteristics. The blend may be formulated into various oral delivery forms, including but not limited to capsules, tablets, soft gels, powders, granules, and chewable or dissolvable tablets, ensuring flexible and convenient consumption and dosing. The patent encompasses all dosages of the active ingredients, allowing for varying concentrations to meet specific health and dietary needs. For tablets or chewable forms, suitable binders and excipients may be included to maintain product stability, while for powder forms, the composition may be packaged in single-serving sachets or bulk containers. Additional ingredients, such as flavoring agents, sweeteners, or other excipients, may be added to enhance consumer acceptability and adherence to different dosage forms and regimens.

### BENEFITS AND APPLICATIONS

The nutritional supplement composition offers multiple benefits to consumers, including:
Support for digestive health and gut microbiota balance due to the properties of the tamarind seed powder working synergistically with pea protein powder.
Suitable for individuals with dietary restrictions or preferences, such as vegetarians, vegans, and those with lactose intolerance or gluten sensitivity.

1. A dietary supplement composition consisting of whole tamarind seed powder and pea protein powder.

2. A dietary supplement composition consisting of whole tamarind seed powder and pea protein powder, administered orally, designed to promote digestive health and overall wellness.

3. A dietary supplement composition of claim 1 or 2, mixed with one or more acceptable excipients and/or one or more further active ingredients.
